(19) United States
(12) Patent Application Publication
(10) Pub. No.: US 2005/0245862 A1
(43) Pub. Date: Nov. 3, 2005

(54) Torque mechanism for interventional catheters

(75) Inventors: Kirk Patrick Seward, Dublin, CA (US); Isidro M. Gandionco, Fremont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: EndoBionics, Inc., San Leandro, CA (US)

(21) Appl. No.: 11/112,871

(22) Filed: Apr. 21, 2005

Related U.S. Application Data
(60) Provisional application No. 60/566,319, filed on Apr. 28, 2004.

(51) Int. Cl. A61M 25/01

(52) U.S. Cl. 604/95.04; 604/528

(57) ABSTRACT

A torque mechanism for interventional catheters provides direct control over rotary positioning of the distal end of a catheter by rotating a proximal handle of the catheter. The torque mechanism comprises a stiff tube within and running the length of a flexible catheter body tube. The stiff tube is bonded at its distal end to the catheter body tubing and at its proximal end to a rotary handle. Catheter body tubing is bonded at its proximal end to a catheter hub. The catheter hub and rotary handle rotate independently, thus torque and position are transmitted by the torque tube while the catheter tube acts as a torsional spring.

### CROSS-REFERENCES TO RELATED APPLICATIONS

The present application is a non-provisional of U.S. Patent Application Ser. No. 60/566,319 (Attorney Docket No. 021621-002100), filed Apr. 28, 2004, the full disclosure of which is incorporated herein by reference.

### BACKGROUND OF THE INVENTION

1. Field of the Invention

The present invention relates generally to medical methods and devices. More particularly, the present invention relates to mechanisms for providing torque between the proximal and distal segments of intravascular catheters, to enable radial positioning of catheter tools within the vasculature.

Coronary artery disease is the leading cause of death and morbidity in the United States and other western societies. In particular, atherosclerosis in the coronary arteries can cause myocardial infarction, commonly referred to as a heart attack, which can be immediately fatal or, even if survived, can cause damage to the heart which can incapacitate the patient. Other coronary diseases which cause death and incapacitation include congestive heart failure, vulnerable or unstable plaque, and cardiac arrhythmias. In addition to coronary artery disease, diseases of the peripheral vasculature can also be fatal or incapacitating. Vascular occlusions, blood clots and thrombus may occlude peripheral blood flow, leading to tissue and organ necrosis. Deep vein thrombosis in the legs can, in the worst cases, require amputation. Clots in the carotid artery can embolize and travel to the brain, potentially causing ischemic stroke.

Percutaneous interventional procedures are very common in the United States and other countries around the world. Intravascular catheter systems are used for procedures such as balloon angioplasty, stent placement, atherectomy, retrieval of blood clots, photodynamic therapy, and drug delivery. All of these procedures involve the placement of long, slender tubes into arteries or veins in order to provide access to the deep recesses of the body without the necessity of open surgery.

While many of the current systems are rotationally symmetrical and would not benefit from any precision in rotary directional placement, several of the systems are meant to be highly directional, for instance to remove eccentric lesions or direct laser energy or drugs toward one side of the artery or vein as necessary. Even in the case of non-directional catheters, the benefit of torqueability or rotatability of the distal end of the catheter inside the body can provide the ability to reduce friction while introducing the catheter into the body or to aid in steering the catheter into the appropriate blood vessel.

Many intravascular catheters are made of polymer materials that creep over time and therefore can take on a shape-set while they are in their package on the shelf. The shape set typically results in somewhat of a curved profile along the length of the catheter. When these catheters are introduced into vessels that have some curvature, they cannot be easily torqued with any precision because the shape-set of the polymer materials accommodates the curvature of the vessel; thus when the catheter is twisted from its proximal end, rather than controlled motion at the distal end, the catheter “snaps” into place. The snapping effect, or catch-up, results in the catheter always having approximately the same rotational direction in the blood vessel, in other words, it always tends to rotate to some multiple of 360° from its original orientation.

Friction adds to the difficulty with torquing of intravascular catheters. When the entire catheter is torqued (as when the proximal end and distal end are linked continuously by the catheter shaft) friction between the catheter shaft and the wall of guiding catheters or blood vessels leads to periods of “stiction” followed by catch-up.

A primary tradeoff in the design of a torque mechanism for interventional catheters is the desire for increased torqueability with the desire for catheter flexibility. During the introduction of a catheter into the blood vessels of a patient, the flexibility of the catheter dictates the amount of tortuosity through which the catheter can be threaded to reach deep into the vasculature. The design of torqueable catheters typically entails the placement of stiff material away from the axis of rotation, increasing the polar moment of inertia along the catheter body by an exponential factor of four for any given increase in distance from the central axis. This is exactly traded off with respect to the second moment of inertia, which dictates that the flexibility of the catheter decreases by the same exponential factor of four as stiff material is located far from the central axis of the catheter.

For this reason, it is desirable to use a material with a variable modulus of elasticity given high amounts of stress or strain. Superelastic shape memory alloys are especially desirable because they resist permanent deformation at up to 10% strains, and their modulus of elasticity can decrease ten-fold when they encounter stress and strain above a certain point (typically around 400 MPa and 3%, respectively). Because these stresses and strains are more easily encountered during bending of the superelastic alloy than torquing of the superelastic alloy, the bending modulus of elasticity can fall locally at a tortuous portion of vessel to accommodate flexibility while maintaining torqueability along most of the catheter.

Another tradeoff of particular interest to the present invention is “real estate” available in the cross-section of an interventional catheter. Most interventional catheters, especially those designed for cardiac applications, are less than 2 mm in diameter. The delivery of fluids or small catheter tools through lumens of the catheter is necessary, but the structure of the catheter body must be retained. While torque wires can be used to provide torsion between the proximal and distal end of the catheter, torque tubing allows for the placement of material away from the catheter's central axis of rotation and provides for a fluid or tool delivery channel. Fluid delivery velocity through a channel is dictated by pressure (to the first power), fluid viscosity (to the first power), and channel size (to the fourth power). Thus, as the channel diameter is increased, fluid can more easily be passed from proximal to distal end. The desire to deliver fluids, therefore, is a second trade-off against the flexibility of the catheter. Torque tubing surrounding the catheter has been disclosed in the prior art, but these designs often lead to catheters that are too stiff to introduce into complex vasculature. In these cases, the torque tubing typically does not extend to the distal aspects of the catheter, leading to limited torque control over the distal tip.

Of particular interest to the present invention, catheters carrying microneedles capable of delivering therapeutic and other agents deep into the adventitial layer surrounding blood vessel lumens have been described U.S. Pat. No. 6,547,803, issued on Apr. 15, 2003, and in co-pending application Ser. No. 09/961,080, filed on Sep. 20, 2001, and Ser. No. 09/961,079, also filed on Sep. 20, 2001, all of which have common inventorship with but different assignment than the present application, the full disclosures of which are incorporated herein by reference.

The designs described in the issued patent and copending applications have numerous advantages. The microneedles are delivered in a direction which is substantially perpendicular to the axis of the catheter, thus maximizing the depth of needle penetration into the wall and reducing trauma and injury. Moreover, by locating the needles on the exterior of an expanding involuted surface, the needles can be injected into tissue fully up to their point of attachment to the catheter, further maximizing the needle penetration depth which may be achieved. In the case of these issued and copending needle-deployment catheter applications, an integrated torque mechanism provides the ability to direct the needle in a particular direction once the catheter is placed into the vasculature.

For these reasons, it would be desirable to provide improved methods for transmitting torque between the proximal end of catheters and distal catheter tips in catheter designs. It would be particularly advantageous if these methods were useful for providing torque from the proximal to distal end while avoiding “snap” or catch-up. It is a particular objective of the present invention to provide methods and structures that optimize torqueability, flexibility, and “real-estate” efficiency so that the catheter may be rotationally directed, delivered into complex vasculature, and deliver fluids or small tools via a channel through the torque mechanism. It is a further objective that the methods be simple and economic to implement and be useful with a wide range of vascular and other medical catheters. At least some of these objectives will be met by the inventions described hereinafter.

2. Description of the Background Art

U.S. Pat. No. 5,114,407 describes a catheter having a torque wire running the length of a catheter body, with attachments at the proximal and distal end to facilitate torquing of the distal tip of the catheter. U.S. Pat. Nos. 6,611,720; 6,287,301; 6,246,914; 5,328,467; 4,998,917; and 4,790,831 describe a number of specific catheter torque mechanism constructions.

### BRIEF SUMMARY OF THE INVENTION

In a first aspect of the present invention, a catheter comprises a tubular catheter body with a proximal and distal end and a torque mechanism contained within the tubular catheter body. The torque mechanism is a tubular member with an inner channel capable of transferring fluids or small catheter tools. The tubular catheter body may contain more than one lumen. Within at least one lumen of the catheter body is contained the torque tube. The torque tube is generally stiffer material than the tubular catheter body, such that the torque tube can transmit torque independently of the catheter body tubing. The torque tube is affixed to the distal end of the catheter body, but not to the proximal end of the catheter body.

The proximal end of the torque tube is affixed to a rotary handle that can be rotated independently from the proximal end of the catheter body tubing. The torque tube generally runs nearly the entire length of the catheter body tubing. The torque tube may extend proximally some distance from the catheter body tubing to enable the placement of the rotary handle.

In an exemplary embodiment, the torque tubing is made of a shape memory alloy such as nitinol and the catheter body tubing is made of a polymer such as polyether block amide (Pebax). The torque tubing is affixed to the distal end of the catheter body tubing by adhesive bonding such as with cyanoacrylate adhesive or by fusion bonding such as by melting the catheter body tubing around the torque tubing. The distal end of the torque tubing may be shaped or have fins to enhance a bond to the distal end of the catheter body tubing. Distal of the bond joint between the catheter body and the torque tubing may be an active catheter component, and further distal of the active component may be a catheter distal tip, which facilitates guidewire tracking of the catheter into the vasculature.

In a further exemplary embodiment, the proximal end of the torque tubing is free to rotate within the catheter body tubing, but affixed to a rotary handle, by similar means as the bond between the distal end of the torque tubing and the catheter body tubing. The catheter body tubing is affixed to a hub at its proximal end. The rotary handle will usually have a locking mechanism in place at the interface between the handle and the hub of the catheter body tubing.

The locking mechanism between the catheter body hub and the rotary handle may take the form of a mechanical spring-loaded ratchet-like interface, such that the rotary handle can be rotated to discrete angles with respect to the catheter hub. The locking mechanism may alternatively take the form of a friction interface, such as by the presence of a silicone ring around a segment of the rotary handle that is inserted with some degree of interference into the catheter hub. The friction interface does not provide for discrete stops, but continuous motion with the ability to stop the rotation between the handle and the hub at any angle.

In the embodiment described above, as the rotary handle is turned with respect to the catheter hub, the torque tubing transmits torque to the distal end of the catheter, turning the distal tip. Because the torque tubing is elastic, some deformation will occur between the proximal and distal ends of the torque tubing. The distal rotation will therefore experience some gain with respect to the rotation of the handle. For example, one proximal handle rotation may lead to one-tenth to one distal tip rotation. The amount of rotation of the distal tip with respect to the rotary handle is dictated by the tortuosity of the catheter placement, the friction between the torque tube and the catheter body, and the relative flexibility between the torque tube and the catheter body. As the torque tube becomes much stiffer than the catheter body, the transmitted torque becomes closer to one-to-one transmission between proximal and distal ends. Also, as the catheter is straighter and encounters less friction between proximal and distal ends, the transmitted torque approaches one-to-one. The flexibility of the catheter body tubing resists excessive rotation of the torque handle with respect to the hub, acting as a torsional spring, and can be used to guide the distal tip back to its original rotation.

In a still further exemplary embodiment, a bearing surface may be incorporated between the torque tubing and the catheter body to reduce the friction between the two surfaces. The bearing surface will usually be incorporated as a freestanding tube, unaffixed to either the catheter body or the torque tubing. The bearing tube will usually be made of a polymer such as polyimide, polyethylene, polypropylene, polyethyl teraphthalate (PET) or PTFE (Teflon©).

### BRIEF DESCRIPTION OF THE DRAWINGS

FIG. 1A is a schematic, perspective view of an intravascular injection catheter suitable for use in the methods and systems of the present invention.

FIG. 1B is a cross-sectional view along line 1B-1B of FIG. 1A.

FIG. 1C is a cross-sectional view along line 1C-1C of FIG. 1A.

FIG. 2A is a schematic, perspective view of the catheter of FIGS. 1A-1C shown with the injection needle deployed.

FIG. 2B is a cross-sectional view along line 2B-2B of FIG. 2A.

FIG. 3 is a schematic, perspective view of the intravascular catheter of FIGS. 1A-1C injecting therapeutic cells into an adventitial space surrounding a coronary blood vessel in accordance with the methods of the present invention.

FIG. 4 is a schematic, perspective view of another embodiment of an intravascular injection catheter useful in the methods of the present invention.

FIG. 5 is a schematic, perspective view of still another embodiment of an intravascular injection catheter useful in the methods of the present invention, as inserted into a patient's vasculature.

FIG. 6 is a perspective view of a needle injection catheter useful in the methods and systems of the present invention.

FIG. 7 is a cross-sectional view of the catheter FIG. 6 shown with the injection needle in a retracted configuration.

FIG. 8 is a cross-sectional view similar to FIG. 7, shown with the injection needle laterally advanced into luminal tissue for the delivery of therapeutic cells according to the present invention.

FIG. 9 is a perspective view of a catheter containing a torque mechanism as described in the present invention. The catheter in this view does not depict the attached distal tool, such as the needle injection tools of FIG. 1 through 8.

FIG. 10 is a cross-sectional view of FIG. 9, showing syringes and stopcocks attached to a catheter with an integrated rotary handle and torque tube.

FIG. 11 is a cross-sectional view of a rotary handle and catheter hub representative of the present invention.

FIG. 12 is a cross-sectional view of the distal bond between the torque tube and the catheter body, along with a representation of a bearing tube.

FIG. 13 is a perspective, partial cut-away view of a catheter employing the torque mechanism of the present invention. FIG. 13 displays the distal attachment between the torque tube and the catheter body.

FIG. 14 is a representative cross-section of a catheter body shaft that could be used when employing the torque mechanism of the present invention.

### DETAILED DESCRIPTION OF THE INVENTION

By way of example, the first eight figures illustrate a needle injection catheter that can benefit from the directability offered by the torque mechanism of the present invention. The final six figures illustrate the torque mechanism that may be integrated with a catheter such as the needle injection catheter.

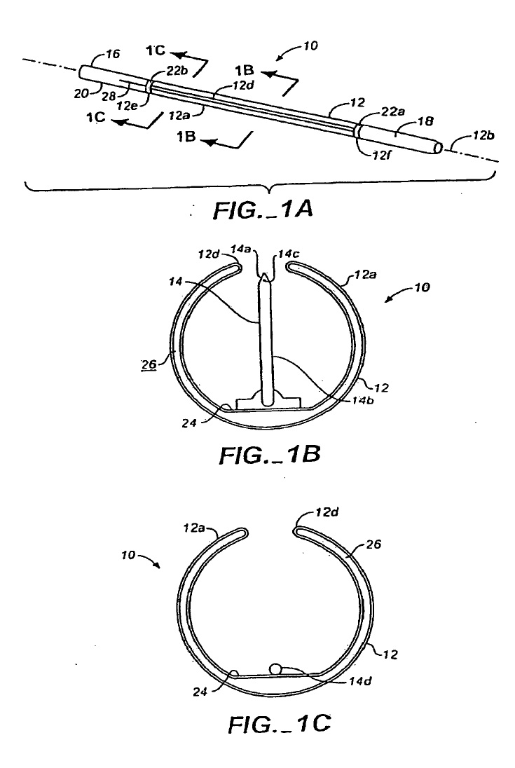

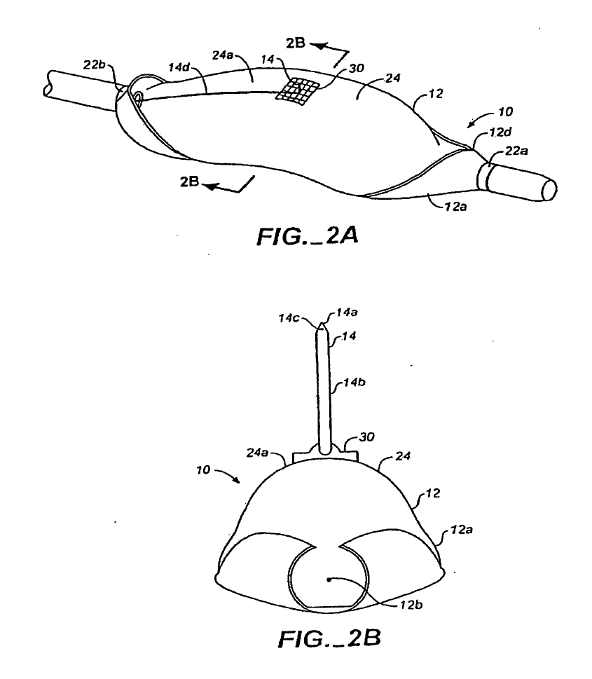

As shown in FIGS. 1A-2B, a microfabricated intravascular catheter 10 includes an actuator 12 having an actuator body 12a and central longitudinal axis 12b. The actuator body more or less forms a C-shaped outline having an opening or slit 12d extending substantially along its length. A microneedle 14 is located within the actuator body, as discussed in more detail below, when the actuator is in its unactuated condition (furled state) (FIG. 1B). The microneedle is moved outside the actuator body when the actuator is operated to be in its actuated condition (unfurled state) (FIG. 2B).

The actuator may be capped at its proximal end 12e and distal end 12f by a lead end 16 and a tip end 18, respectively, of a therapeutic catheter 20. The catheter tip end serves as a means of locating the actuator inside a blood vessel by use of a radio opaque coatings or markers. The catheter tip also forms a seal at the distal end 12f of the actuator. The lead end of the catheter provides the necessary interconnects (fluidic, mechanical, electrical or optical) at the proximal end 12e of the actuator.

Retaining rings 22a and 22b are located at the distal and proximal ends, respectively, of the actuator. The catheter tip is joined to the retaining ring 22a, while the catheter lead is joined to retaining ring 22b. The retaining rings are made of a thin, on the order of 10 to 100 microns (μm), substantially rigid material, such as Parylene (types C, D or N), or a metal, for example, aluminum, stainless steel, gold, titanium or tungsten. The retaining rings form a rigid substantially “C”-shaped structure at each end of the actuator. The catheter may be joined to the retaining rings by, for example, a butt-weld, an ultra sonic weld, integral polymer encapsulation or an adhesive such as an epoxy.

The actuator body further comprises a central, expandable section 24 located between retaining rings 22a and 22b. The expandable section 24 includes an interior open area 26 for rapid expansion when an activating fluid is supplied to that area. The central section 24 is made of a thin, semi-rigid or rigid, expandable material, such as a polymer, for instance, Parylene (types C, D or N), silicone, polyurethane or polyimide. The central section 24, upon actuation, is expandable somewhat like a balloon-device.

The central section is capable of withstanding pressures of up to about 100 psi upon application of the activating fluid to the open area 26. The material from which the central section is made of is rigid or semi-rigid in that the central section returns substantially to its original configuration and orientation (the unactuated condition) when the activating fluid is removed from the open area 26. Thus, in this sense, the central section is very much unlike a balloon which has no inherently stable structure.

The open area 26 of the actuator is connected to a delivery conduit, tube or fluid pathway 28 that extends from the catheter's lead end to the actuator's proximal end. The activating fluid is supplied to the open area via the delivery tube. The delivery tube may be constructed of Teflon© or other inert plastics. The activating fluid may be a saline solution or a radio-opaque dye.

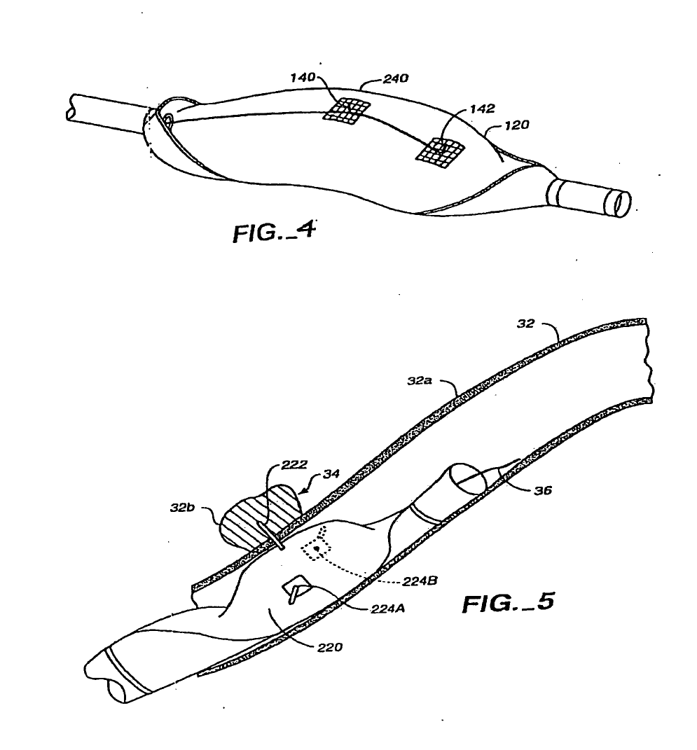

The microneedle 14 may be located approximately in the middle of the central section 24. However, as discussed below, this is not necessary, especially when multiple microneedles are used. The microneedle is affixed to an exterior surface 24a of the central section. The microneedle is affixed to the surface 24a by an adhesive, such as cyanoacrylate. Alternatively, the microneedle maybe joined to the surface 24a by a metallic or polymer mesh-like structure 30 (See FIG. 4F), which is itself affixed to the surface 24a by an adhesive. The mesh-like structure may be-made of, for instance, steel or nylon.

The microneedle includes a sharp tip 14a and a shaft 14b. The microneedle tip can provide an insertion edge or point. The shaft 14b can be hollow and the tip can have an outlet port 14c, permitting the injection of a pharmaceutical or drug into a patient. The microneedle, however, does not need to be hollow, as it may be configured like a neural probe to accomplish other tasks.

As shown, the microneedle extends approximately perpendicularly from surface 24a. Thus, as described, the microneedle will move substantially perpendicularly to an axis of a vessel or artery into which has been inserted, to allow direct puncture or breach of vascular walls.

The microneedle further includes a pharmaceutical or drug supply conduit, tube or fluid pathway 14d which places the microneedle in fluid communication with the appropriate fluid interconnect at the catheter lead end. This supply tube may be formed integrally with the shaft 14b, or it may be formed as a separate piece that is later joined to the shaft by, for example, an adhesive such as an epoxy.

The needle 14 may be a 30-gauge, or smaller, steel needle. Alternatively, the microneedle may be microfabricated from polymers, other metals, metal alloys or semiconductor materials. The needle, for example, may be made of Parylene, silicon or glass. Microneedles and methods of fabrication are described in U.S. application Ser. No. 09/877,653, filed Jun. 8, 2001, entitled “Microfabricated Surgical Device”, which has common inventorship with but different assignment than the present application, the entire disclosure of which is incorporated herein by reference.

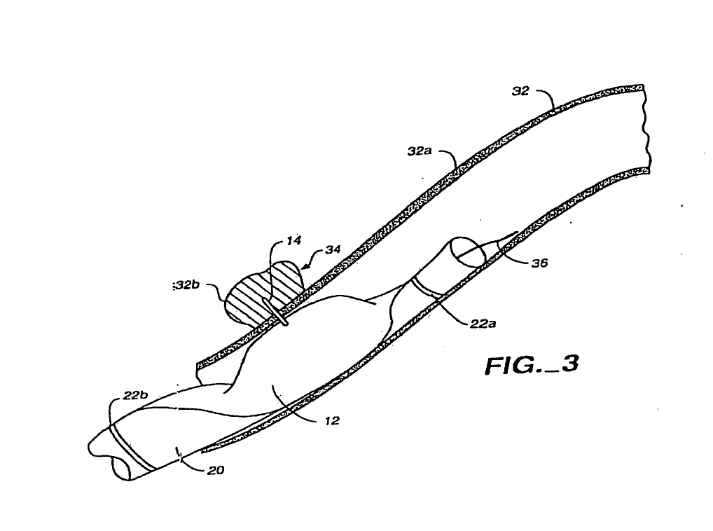

The catheter 20, in use, is inserted through an artery or vein and moved within a patient's vasculature, for instance, a vein 32, until a specific, targeted region 34 is reached (see FIG. 3). The targeted region 34 may be the site of tissue damage or more usually will be adjacent the sites typically being within 100 mm or less to allow migration of the cells. As is well known in catheter-based interventional procedures, the catheter 20 may follow a guide wire 36 that has previously been inserted into the patient. Optionally, the catheter 20 may also follow the path of a previously-inserted guide catheter (not shown) that encompasses the guide wire.

During maneuvering of the catheter 20, well-known methods of fluoroscopy or magnetic resonance imaging (MRI) can be used to image the catheter and assist in positioning the actuator 12 and the microneedle 14 at the target region. As the catheter is guided inside the patient's body, the microneedle remains furled or held inside the actuator body so that no trauma is caused to the vascular walls.

After being positioned at the target region 34, movement of the catheter is terminated and the activating fluid is supplied to the open area 26 of the actuator, causing the expandable section 24 to rapidly unfurl, moving the microneedle 14 in a substantially perpendicular direction, relative to the longitudinal central axis 12b of the actuator body 12a, to puncture a vascular wall 32a. It may take only between approximately 100 milliseconds and two seconds for the microneedle to move from its furled state to its unfurled state.

The ends of the actuator at the retaining rings 22a and 22b remain rigidly fixed to the catheter 20. Thus, they do not deform during actuation. Since the actuator begins as a furled structure, its so-called pregnant shape exists as an unstable buckling mode. This instability, upon actuation, produces a large-scale motion of the microneedle approximately perpendicular to the central axis of the actuator body, causing a rapid puncture of the vascular wall without a large momentum transfer. As a result, a microscale opening is produced with very minimal damage to the surrounding tissue. Also, since the momentum transfer is relatively small, only a negligible bias force is required to hold the catheter and actuator in place during actuation and puncture.

The microneedle, in fact, travels so quickly and with such force that it can enter perivascular tissue 32b as well as vascular tissue. Additionally, since the actuator is “parked” or stopped prior to actuation, more precise placement and control over penetration of the vascular wall are obtained.

After actuation of the microneedle and delivery of the cells to the target region via the microneedle, the activating fluid is exhausted from the open area 26 of the actuator, causing the expandable section 24 to return to its original, furled state. This also causes the microneedle to be withdrawn from the vascular wall. The microneedle, being withdrawn, is once again sheathed by the actuator.

Various microfabricated devices can be integrated into the needle, actuator and catheter for metering flows, capturing samples of biological tissue, and measuring pH. The device 10, for instance, could include electrical sensors for measuring the flow through the microneedle as well as the pH of the pharmaceutical being deployed. The device 10 could also include an intravascular ultrasonic sensor (IVUS) for locating vessel walls, and fiber optics, as is well known in the art, for viewing the target region. For such complete systems, high integrity electrical, mechanical and fluid connections are provided to transfer power, energy, and pharmaceuticals or biological agents with reliability.

By way of example, the microneedle may have an overall length of between about 200 and 3,000 microns (μm). The interior cross-sectional dimension of the shaft 14b and supply tube 14d may be on the order of 20 to 250 um, while the tube's and shaft's exterior cross-sectional dimension may be between about 100 and 500 μm. The overall length of the actuator body may be between about 5 and 50 millimeters (mm), while the exterior and interior cross-sectional dimensions of the actuator body can be between about 0.4 and 4 mm, and 0.5 and 5 mm, respectively. The gap or slit through which the central section of the actuator unfurls may have a length of about 4-40 mm, and a cross-sectional dimension of about 50-500 μm. The diameter of the delivery tube for the activating fluid may be about 100 μm. The catheter size may be between 1.5 and 15 French (Fr).

Variations of the invention include a multiple-buckling actuator with a single supply tube for the activating fluid. The multiple-buckling actuator includes multiple needles that can be inserted into or through a vessel wall for providing injection at different locations or times.

For instance, as shown in FIG. 4, the actuator 120 includes microneedles 140 and 142 located at different points along a length or longitudinal dimension of the central, expandable section 240. The operating pressure of the activating fluid is selected so that the microneedles move at the same time. Alternatively, the pressure of the activating fluid may be selected so that the microneedle 140 moves before the microneedle 142.

Specifically, the microneedle 140 is located at a portion of the expandable section 240 (lower activation pressure) that, for the same activating fluid pressure, will buckle outwardly before that portion of the expandable section (higher activation pressure) where the microneedle 142 is located. Thus, for example, if the operating pressure of the activating fluid within the open area of the expandable section 240 is two pounds per square inch (psi), the microneedle 140 will move before the microneedle 142. It is only when the operating pressure is increased to four psi, for instance, that the microneedle 142 will move. Thus, this mode of operation provides staged buckling with the microneedle 140 moving at time t₁, and pressure p₁, and the microneedle 142 moving at time t₂ and p₂, with t₁, and p₁, being less than t₂ and p₂, respectively.

This sort of staged buckling can also be provided with different pneumatic or hydraulic connections at different parts of the central section 240 in which each part includes an individual microneedle.

Also, as shown in FIG. 5, an actuator 220 could be constructed such that its needles 222 and 224A move in different directions. As shown, upon actuation, the needles move at angle of approximately 90° to each other to puncture different parts of a vessel wall. A needle 224B (as shown in phantom) could alternatively be arranged to move at angle of about 180° to the needle 224A.

The above catheter designs and variations thereon, are described in published U.S. patent application Nos. 2003/005546 and 2003/0055400, the full disclosures of which are incorporated herein by reference. Co-pending application Ser. No. 10/350,314, assigned to the assignee of the present application, describes the ability of substances delivered by direct injection into the adventitial and pericardial tissues of the heart to rapidly and evenly distribute within the heart tissues, even to locations remote from the site of injection. The full disclosure of that co-pending application is also incorporated herein by reference. An alternative needle catheter design suitable for delivering the therapeutic cells of the present invention will be described below. That particular catheter design is described and claimed in co-pending application Ser. No. 10/397,700 (Attorney Docket No.021621-001500 U.S.), filed on Mar. 19, 2003, the full disclosure of which is incorporated herein by reference.

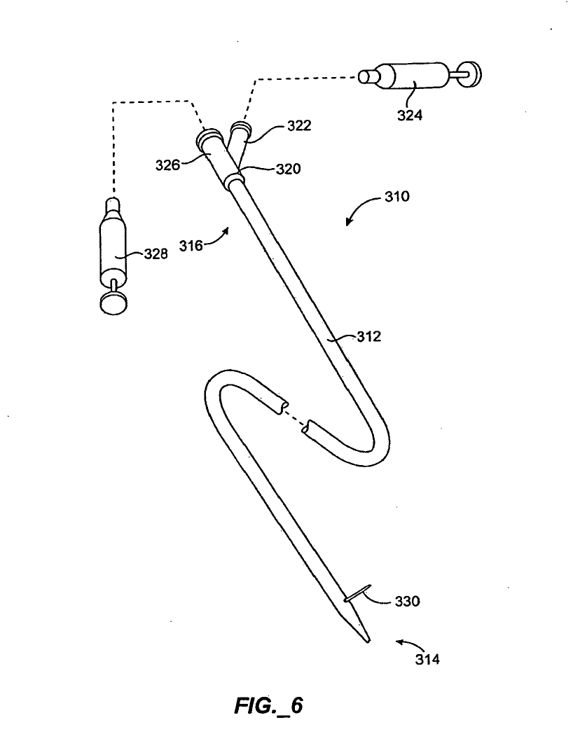

Referring now to FIG. 6, a needle injection catheter 310 constructed in accordance with the principles of the present invention comprises a catheter body 312 having a distal end 314 and a proximal 316. Usually, a guide wire lumen 313 will be provided in a distal nose 352 of the catheter, although over-the-wire and embodiments which do not require guide wire placement will also be within the scope of the present invention. A two-port hub 320 is attached to the proximal end 316 of the catheter body 312 and includes a first port 322 for delivery of a hydraulic fluid, e.g., using a syringe 324, and a second port 326 for delivering the pharmaceutical agent, e.g., using a syringe 328. A reciprocatable, deflectable needle 330 is mounted near the distal end of the catheter body 312 and is shown in its laterally advanced configuration in FIG. 6.

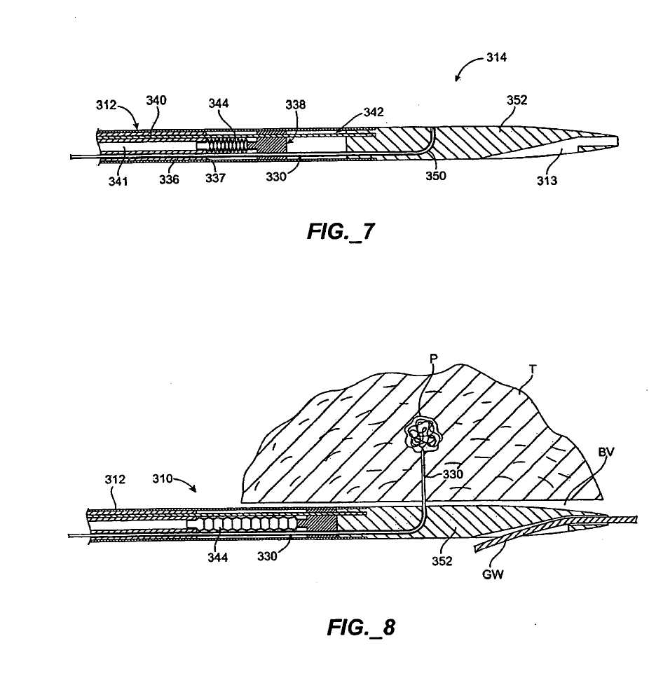

Referring now to FIG. 7, the proximal end 314 of the catheter body 312 has a main lumen 336 which holds the needle 330, a reciprocatable piston 338, and a hydraulic fluid delivery tube 340. The piston 338 is mounted to slide over a rail 342 and is fixedly attached to the needle 330. Thus, by delivering a pressurized hydraulic fluid through a lumen 341 tube 340 into a bellows structure 344, the piston 338 may be advanced axially toward the distal tip in order to cause the needle to pass through a deflection path 350 formed in a catheter nose 352.

As can be seen in FIG. 8, the catheter 310 may be positioned in a coronary blood vessel BV, over a guide wire GW in a conventional manner. Distal advancement of the piston 338 causes the needle 330 to advance into luminal tissue T adjacent to the catheter when it is present in the blood vessel. The therapeutic cells may then be introduced through the port 326 using syringe 328 in order to introduce a plume P of agent in the cardiac tissue, as illustrated in FIG. 8. The plume P will be within or adjacent to the region of tissue damage as described above.

The needle 330 may extend the entire length of the catheter body 312 or, more usually, will extend only partially in therapeutic cells delivery lumen 337 in the tube 340. A proximal end of the needle can form a sliding seal with the lumen 337 to permit pressurized delivery of the agent through the needle.

The needle 330 will be composed of an elastic material, typically an elastic or super elastic metal, typically being nitinol or other super elastic metal. Alternatively, the needle 330 could be formed from a non-elastically deformable or malleable metal which is shaped as it passes through a deflection path. The use of non-elastically deformable metals, however, is less preferred since such metals will generally not retain their straightened configuration after they pass through the deflection path.

The bellows structure 344 may be made by depositing by parylene or another conformal polymer layer onto a mandrel and then dissolving the mandrel from within the polymer shell structure. Alternatively, the bellows 344 could be made from an elastomeric material to form a balloon structure. In a still further alternative, a spring structure can be utilized in, on, or over the bellows in order to drive the bellows to a closed position in the absence of pressurized hydraulic fluid therein.

After the therapeutic cells are delivered through the needle 330, as shown in FIG. 8, the needle is retracted and the catheter either repositioned for further agent delivery or withdrawn. In some embodiments, the needle will be retracted simply by aspirating the hydraulic fluid from the bellows 344. In other embodiments, needle retraction may be assisted by a return spring, e.g., locked between a distal face of the piston 338 and a proximal wall of the distal tip 352 (not shown) and/or by a pull wire attached to the piston and running through lumen 341.

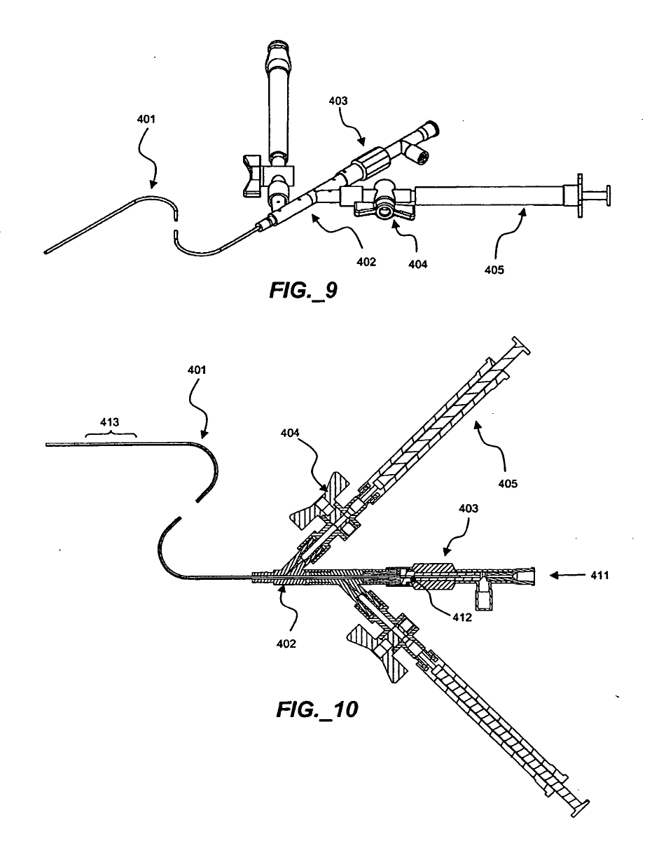

Referring now to FIG. 9, a torque mechanism is integrated into a catheter by bonding it distally into a catheter body 401 and proximally into a rotary handle 403. The rotary handle rotates freely or with discrete or continuous engagement with respect to a hub 402. Other attachments may be made to the hub, such as stopcocks 404 or syringes 405. Additional components may also be included such as is shown proximally of the handle 403.

In FIG. 10, a cross sectional view of the catheter of FIG. 9 is displayed. Additional detail is provided of the distal bonding section 413, the engagement between the hub and the rotary handle 412, and the proximal fluidic port 411 that connects to the torque tube. The torque tube is bonded into the rotary handle 403 and at the distal end of the catheter body 401 over a length shown by 413. Such bond lengths may range from 5 to 50 mm, and are preferably around 25 mm. The length of the catheter body 401 is typically around 140 cm, but may be more or less depending on the application for which the catheter is intended. An active distal component, such as one of the needle injection devices described above, is not displayed in FIG. 9 or 10, but would simply be attached at the distal end of the catheter body tubing 401.

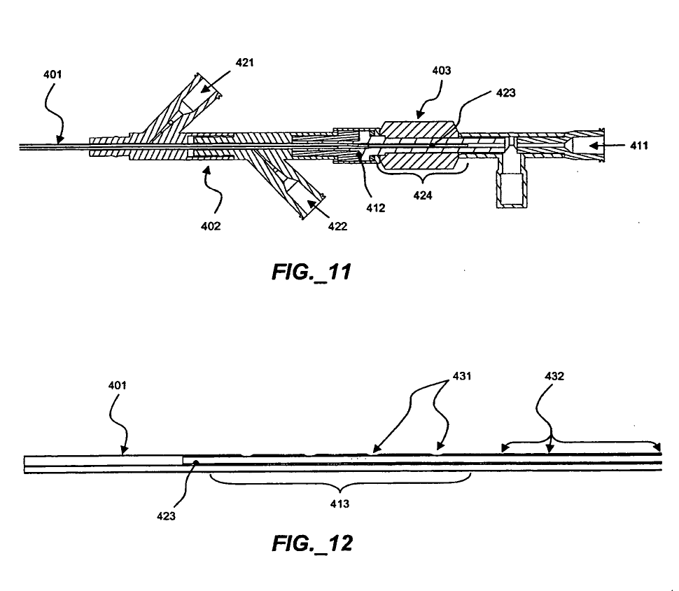

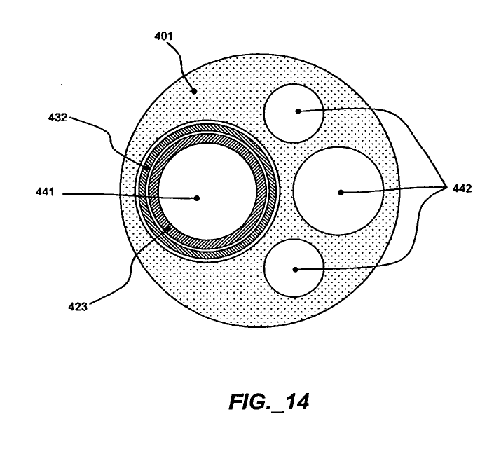

The proximal end of the catheter is illustrated in greater cross-sectional detail in FIG. 11. Here, additional fluidic sideports 421 and 422 are seen as part of the catheter hub 402. These sideports may route into additional lumens of the catheter, as can be seen in FIG. 14. The engagement interface 412 between the rotary handle 403 and the hub 402 is more clearly illustrated. The engagement interface may be made at a 90° angle as is shown here or could be at an angle between 90° and 0°. The engagement interface may be made by interference-fitting ratcheted surfaces, slippery, low friction surfaces, or high friction surfaces such as a silicone-on-plastic interface.

As can also be seen in FIG. 11, the torque tube 423 has a bond interface with the rotary handle 403 that may run the length indicated by 424. The bond interface between the torque tube and the handle may be an interference bond, a mechanical bond, such as fusion bonding, or a chemical bond, such as cyanoacrylate adhesion.

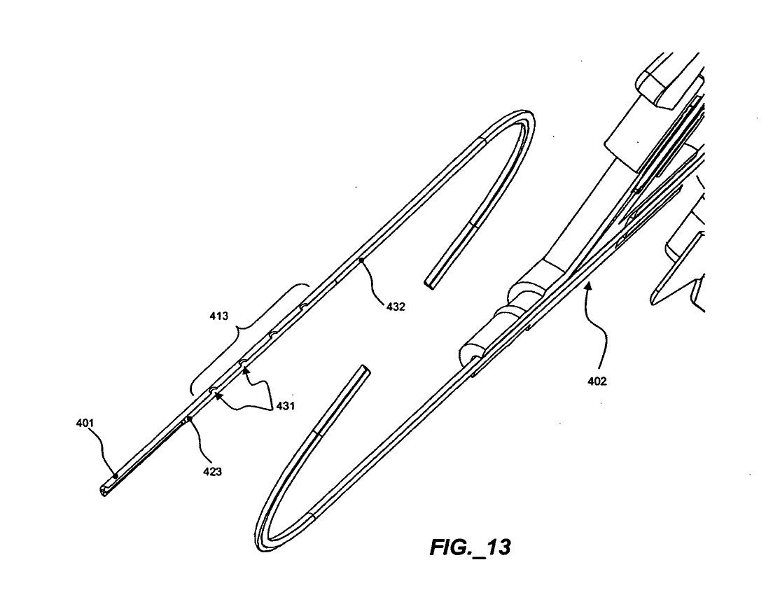

The distal bond interface between the torque tubing and the catheter body tubing is displayed in FIG. 12. In this embodiment, the torque tube 423 terminates just proximal of the distal end o the catheter body tubing 401. The bond between the torque tubing and the catheter body tubing can be formed over a length 413 by introducing adhesive into skive holes 431 made in the catheter body 401.

Proximal of the bond interface, the bearing tube 432 may be implemented into the torque mechanism. The bearing tube may be bonded to either the catheter body 401 or the torque tube 423 or may be free-floating, which results in the least friction between all surfaces and the best bearing configuration. The bearing tube 432 may run the length of the catheter from just proximal of the distal bond 413 to just distal of the proximal bond 424.

The torque tube 423, bearing tube 432, distal bond length 413, catheter body 401, skive holes 431, and catheter hub 402 are also displayed in the cut-away view of FIG. 13. In this view, all components except the torque tube 423 and bearing tube 432 are cut away to reveal the torque and bearing tubes inside the lumen of the catheter body.

Referring now to FIG. 14, a cross-sectional view of the catheter body tubing 401 could reveal not only the lumen 441 through which the torque tube 423 and the bearing tube 432 are routed, but additional lumens 442, through which other fluids or tools such as guidewires or fiber optics could be routed. The number of additional lumens could range from zero to more than ten, but will most often be in the range of one to five. The bearing tube 432 provides a low-friction interface between the torque tube 423 and the body tubing 401. Fluids or catheter tools may be delivered through the port 411 in FIG. 10 and out the lumen 441 in FIG. 14.

By way of example, the catheter body tubing 401 may have a diameter of 0.5 to 15 mm, but will more typically be in the range from 1 to 4 mm. The torque tubing 423 may be around one quarter to one half the diameter of the catheter shaft, or may be driven by dimensional qualifications with outer diameter between 0.1 and 2 mm, more often in the range of 0.3 to 1 mm. The internal diameter of the torque tubing may be in the range of 0.05 and 1.8 mm, often in the range of 0.1 to 0.8 mm. The bearing tube 432 would often be just larger than the torque tubing, with a clearance between the two tubes of around 0.05 to 0.2 mm and a wall thickness of around 0.01 to 0.10 mm.

While the above is a complete description of the preferred embodiments of the invention, various alternatives, modifications, and equivalents may be used. Therefore, the above description should not be taken as limiting the scope of the invention which is defined by the appended claims.

1. A mechanism for providing rotational control of the distal end of a catheter tubing from the proximal end of the catheter, said mechanism comprising:
a flexible catheter body tubing,
a stiff torque tubing disposed within said catheter body tubing,
a bond joint at the distal aspect of the torque tubing between the torque tubing and the catheter body tubing,
a catheter hub bonded or affixed to the proximal aspect of the catheter body tubing,
a rotary handle bonded or affixed to the proximal aspect of the torque tubing, and
an interface between the catheter hub and the rotary handle such that the rotary handle can be rotated with respect to the catheter hub.

2. A mechanism as in claim 1, wherein the catheter body tubing is a polymer with flexural stiffness less than that of the torque tubing.

3. A mechanism as in claim 1, wherein the torque tubing is a shape memory alloy.

4. A mechanism as in claim 3, wherein the shape memory alloy is primarily composed of nickel and titanium.

5. A mechanism as in claim 3, wherein the shape memory alloy is superelastic at 37° C., or has an austenite finish temperature lower than 37° C.

6. A mechanism as in claim 1, wherein one or more of the joints at which two components are bonded or affixed comprises a mechanical interference joint.

7. A mechanism as in claim 1, wherein one or more of the joints at which two components are bonded or affixed comprises an adhesive bond joint.

8. A mechanism as in claim 1, wherein one or more of the joints at which two components are bonded or affixed comprises a fusion bond joint.

9. A mechanism for providing rotational control of the distal end of a catheter tubing from the proximal end of the catheter, said mechanism comprising:
a flexible catheter body tubing,
a stiff torque tubing disposed within said catheter body tubing,
a bond joint at the distal aspect of the torque tubing between the torque tubing and the catheter body tubing,
a catheter hub bonded or affixed to the proximal aspect of the catheter body tubing,
a rotary handle bonded or affixed to the proximal aspect of the torque tubing, an interface between the catheter hub and the rotary handle such that the rotary handle can be rotated with respect to the catheter hub, and
a bearing tube disposed between the torque tube and the catheter tube for at least a portion of the distance between the distal end of the torque tube and the proximal end of the torque tube.

10. A mechanism as in claim 9, wherein the catheter body tubing is a polymer with flexural stiffness less than that of the torque tubing.

11. A mechanism as in claim 9, wherein the torque tubing is a shape memory alloy.

12. A mechanism as in claim 11, wherein the shape memory alloy is primarily composed of nickel and titanium.

13. A mechanism as in claim 11, wherein the shape memory alloy is superelastic at 37° C., or has an austenite finish temperature lower than 37° C.

14. A mechanism as in claim 9, wherein one or more of the joints at which two components are bonded or affixed comprises a mechanical interference joint.

15. A mechanism as in claim 9, wherein one or more of the joints at which two components are bonded or affixed comprises an adhesive bond joint.

16. A mechanism as in claim 9, wherein one or more of the joints at which two components are bonded or affixed comprises a fusion bond joint.

17. A mechanism as in claim 9, wherein the bearing tube is a polymer.

18. A mechanism as in claim 17, wherein the polymer is chosen from the group: polyimide, PTFE, PET, polyethylene, and polypropylene.
